(19) United States
(12) Patent Application Publication
(10) Pub. No.: US 2003/0082217 A1
(43) Pub. Date: May 1, 2003

(54) Use of heat-stabilizing microcapsules to improve the activity or penetration of cosmetic or pharmaceutical active principles

(76) Inventors: Isabelle Afriat, New York, NY (US); Bruno Biatry, Vincennes (FR)

Correspondence Address: STEPTOE & JOHNSON LLP, 1330 Connecticut Avenue, N.W., Washington, DC, 20036, US

(21) Appl. No.: 09/983,764

(22) Filed: Oct. 25, 2001

(51) Int. Cl. A61K 7/00

(52) U.S. Cl. 424/401

(57) ABSTRACT

The invention relates to topical compositions comprising, in a physiologically acceptable support, a combination

of at least one cosmetic or pharmaceutical active principle and

of microcapsules containing at least one crystalline compound with a heat of fusion (ΔH_{f}), measured by differential thermal analysis, of between 75 and 330 kJ/kg and a melting point of greater than or equal to 30° C.

The present invention relates to the use of heat-stabilizing microcapsules to improve the activity or penetration of cosmetic or pharmaceutical active principles, to topical compositions containing cosmetic or pharmaceutical active principles combined with such microcapsules, and also cosmetic processes for treating the skin using these topical compositions.

In the cosmetic or pharmaceutical field, there are many applications which require a supply of heat.

Specifically, the local production of heat may, for example, promote the cleansing of the skin by an effect of dilating the pores, or reinforce the action of a product, such as a slimming cream, by activating the blood microcirculation or by improving the penetration of the applied active principle into the skin.

This heat may be supplied, for example, by massaging the areas to be treated, which nevertheless requires an occasionally sustained effort that the user is not always prepared to make.

The heat of dilution of concentrated polyethylene glycols is also often used for this purpose. However, this approach has the drawback that direct contact between the chemical product (polyethylene glycol) and the skin may cause skin irritation.

Consequently, there is a need for cosmetic or pharmaceutical compositions enabling a local supply of heat, but which do not involve direct contact of the heating product with the skin and which do not require any massaging.

The Applicant has discovered, surprisingly, that it is possible to solve the problem mentioned above by using specific microcapsules, described in greater detail below, which have heat restituting and absorbing capacities.

The microcapsules under consideration contain, in a leaktight envelope, partially or totally crystalline compounds which, when brought to a temperature close to their melting point, absorb a large amount of heat, known as the latent heat of fusion. The absorption of this latent heat of fusion is reflected by a stability of the temperature of the compound despite it being supplied with heat energy. This effect is similar to a thermal “buffer” effect and makes it possible to thermostatically regulate, for a certain period and over a temperature range close to the melting point of the compound, the immediate environment of the microcapsules despite a variation in the external temperature.

The heat-absorbing capacity described above goes hand in hand with the possibility, exploited in the present invention, of restituting the energy absorbed in the form of latent heat of crystallization. Specifically, when the temperature of such a compound in molten form is lowered below its melting point, a stabilization of the temperature will be observed locally and for a certain period, despite the surrounding cooling. The microcapsules containing the compound in molten form thus constitute a reserve of heat energy.

Microcapsules having the reversible heat restituting and absorbing capacities described above will be referred to hereinbelow as “heat-stabilizing microcapsules”.

Consequently, one subject of the present invention is topical compositions comprising, in a physiologically acceptable support, a combination of at least one cosmetic or pharmaceutical active principle and of microcapsules containing at least one crystalline compound with a heat of fusion (ΔH_{f}), measured by differential thermal analysis, of between 75 and 330 kJ/kg and a melting point of greater than or equal to 300° C.

A subject of the invention is also a topical composition comprising, in a physiologically acceptable support, a combination of at least one pharmaceutical active principle and of microcapsules containing at least one crystalline compound with a heat of fusion (ΔH_{f}), measured by differential thermal analysis, of between 75 and 330 kJ/kg and a melting point of greater than or equal to 30° C., as a medicinal product.

Another subject of the invention is cosmetic treatment processes comprising the sequential or simultaneous application of at least one cosmetic active principle and of microcapsules containing at least one crystalline compound with a heat of fusion (ΔH_{f}), measured by differential thermal analysis, of between 75 and 330 kJ/kg and a melting point of greater than or equal to 30° C., preheated to a temperature above the melting point of the encapsulated crystalline compound.

Finally, a subject of the invention is the use of the microcapsules described above for the manufacture of a product intended to improve the cutaneous activity and/or penetration of a pharmaceutical active principle applied simultaneously or beforehand.

The efficacy of the cosmetic compositions of the present invention depends directly on the heat-absorbing capacity of the encapsulated crystalline compounds and on their melting point.

The heat-absorbing capacity of the microcapsules used is directly proportional to the heat of fusion of the encapsulated crystalline compound. This heat of fusion is measured by differential thermal analysis (Differential Scanning Calorimetry).

The heat of fusion of a compound is the amount of energy required to convert a partially or totally crystalline sample into a totally amorphous sample. The thermogram ΔCp=f(T), in which ΔCp represents the difference in heat capacity of the sample relative to a reference sample undergoing no thermal transition in the range studied, thus has an endothermic signal whose area is proportional to the heat of fusion (ΔH_{f}) of the sample.

The term “crystalline compound” as used in the present patent application includes partially and totally crystalline compounds. The degree of crystallinity of the compounds used is not a deciding factor since the compound has the heat of crystal fusion required for the intended use.

As mentioned, the microcapsules used in the cosmetic compositions of the present invention contain crystalline compounds with a heat of fusion of between 75 and 330 kJ/kg, preferably between 100 kJ/kg and 300 kJ/kg and ideally between 150 and 280 kJ/kg.

The encapsulated crystalline compound must have a melting point (=crystallization temperature) of greater than or equal to 30° C., which is a temperature close to skin temperature.

This is an essential characteristic of the present invention since, specifically, in order to obtain the desired heat-storage effect, the crystallization of the encapsulated crystalline compound must take place at a temperature close to skin temperature and preferably above or equal to this temperature.

Needless to say, the crystalline compound must not have too high a melting point corresponding to a temperature which causes an unpleasant sensation of excessive heat, or even of burning.

The melting point range of the encapsulated crystalline compounds in the heat-stabilizing microcapsules used in the present invention ranges especially from 30 to 45° C. and preferably from 32° C. to 40° C.

Examples of encapsulated crystalline compounds that are suitable for the present invention which may be mentioned include:

aliphatic hydrocarbons, preferably with a linear chain, containing from 13 to 28 carbon atoms and preferably from 19 to 22 carbon atoms,

aromatic hydrocarbons,

saturated or unsaturated C₉₋₂₄ fatty acids, especially capric acid, lauric acid and elaidic acid,

saturated, linear or branched C₁₄₋₃₆ fatty alcohols, and especially myristyl alcohol or hexadecyl-2-eicosanol,

C₁₀₋₂₂ fatty acid esters such as benzoyl stearate, methyl cinnamate, methyl palmitate, isostearyl behenate, di-trimethylolpropane tetralaurate sold under the name Hest® 2T-4L by the company Heteren, and di-trimethylolpropane tetrahydroxystearate sold under the name Hest® 2T-5E-4HS by the company Heteren,

mineral salts containing a large fraction of water of crystallization, such as calcium chloride hexahydrate, sodium sulphate decahydrate, sodium hydrogen phosphate dodecahydrate, sodium thiosulphate pentahydrate and nickel nitrate hexahydrate,

C₁₂₋₁₈ fatty acid triglycerides,

certain silicone waxes such as polydimethylsiloxanes containing behenoxy or stearoxy end groups (INCI: behenoxydimethicone and stearoxydimethicone), polymethylstearyloxy-dimethylsiloxane (INCI: stearic ester dimethicone), polymethylstearyl-dimethylsiloxane (INCI: stearyldimethicone), copolymers containing stearyl methacrylate units with polydimethylsiloxane grafts, polymethyltrifluoromethylalkyl-dimethylsiloxane (INCI:

trifluoromethyl (C₁₋₄ alkyl)-dimethicone),

beeswax derivatives, for instance beeswax esterified with a dimethiconol sold under the name Ultrabee® by the company J W Hanson.

These compounds may be used alone or in the form of a mixture of two or more of them.

Mention may also be made of at least partially crystalline hot-melt polymers with a suitable crystal melting point.

Such polymers are, for example, olefinic homopolymers and copolymers, including polyolefinic waxes, such as ethylene homopolymers, copolymers of ethylene and of propylene, copolymers of ethylene and of octene, copolymers of ethylene and of butene and copolymers of ethylene and of vinyl acetate.

Mention may also be made of poly(alkylene oxides), polyalkyl esters, poly(ε-caprolactones), polyamides, in particular those resulting from the polycondensation of fatty acid dimers, and fluoroolefin copolymers.

Another group of crystalline polymers which may be used is formed by the polymers with crystallizable side chains described in J. Polymer Sci.: Macromol. Rev. 8:117-253 (1974). These are vinyl and/or acrylic polymers or copolymers containing a large fraction, generally at least equal to 50% by weight, of copolymerized units comprising long crystallizable linear aliphatic side chains, or crystallizable fluoro or perfluoro side chains. U.S. Pat. No. 5,156,911 describes the use of such polymers with crystallizable side chains in adhesive assemblies whose adhesive properties vary as a function of the temperature.

The encapsulated crystalline compounds that are preferred for the present invention are aliphatic hydrocarbons with a linear chain containing from 19 to 28 carbon atoms and preferably from 20 to 23 carbon atoms, namely n-nonadecane, n-eicosane, n-heneicosane, n-docosane and n-tricosane.

According to the present invention, the crystalline compounds are preferably encapsulated in a leaktight envelope.

This encapsulation is an essential condition for the reversibility of the fusion/crystallization processes. Specifically, in the absence of a leaktight envelope, the molten compound would diffuse through the membrane and into the cosmetic composition and the thermal effect associated with its crystallization would be greatly reduced, or even eliminated.

For this same reason, the envelope must be solid enough to withstand the shear forces during the application of the composition containing them.

The material constituting the wall of the microcapsules may be chosen from any material conventionally used in the field of microencapsulation. This material may be amorphous, crystalline or semi-crystalline. When it is crystalline or semi-crystalline, it must have a melting point greater than that of the encapsulated crystalline compounds. Moreover, this material must be elastic enough to withstand the variations in the volume of the crystalline compound during the phase transition. Furthermore, it must be inert towards the encapsulated substances and the compounds of the cosmetic or pharmaceutical formulation with which it will be in contact.

According to the chosen process, polymers such as polyamides, polyurethanes, polyureas, polyesters, polycyanoacrylates, urea-formaldehyde or melamine-formaldehyde resins and gelatin/gum arabic systems may be used as materials.

The microcapsules may be prepared according to well-known processes described, for example, in the book entitled “Microencapsulation, Methods and Industrial Applications”, published under the direction of S. Benita, Marcel Dekker (1996). Mention may be made, by way of example, of interfacial polymerization or polycondensation, coacervation, atomization, centrifugal extrusion or microencapsulation on rotary discs.

Heat-stabilizing microcapsules are known and sold, for example, under the name Thermasorb® by the company Frisby Technologies Inc. or under the references 9850K and 9850Q by the company 3M.

These microcapsules are in the form of a fine, fluid, non-film-forming powder. Their use is known, for example, in the field of isothermal clothing and footwear, in microelectronic cooling systems and in the field of packaging.

A non-polymer material may also be used as a compound constituting the wall of the microparticles. Microcapsules based on precipitated, amorphous or hydrated silica or on silica which has been made hydrophobic, sold by the company Phase Change Laboratories under the name AcuTemp®, may be used, for example.

The upper size of the microcapsules with a leaktight envelope used in the present invention is preferably limited, for obvious reasons of visibility, to a few tens or hundreds of micrometres. It is generally preferred to use microcapsules with a mean diameter of between 0.01 and 100 micrometres and better still between 0.05 and 50 micrometres.

The proportion of the microcapsules in the topical compositions of the present invention may vary within a wide range which depends on the formulation and the intended application.

The topical compositions of the present invention generally contain from 0.1% to 95% by weight and preferably from 5% to 90% by weight of heat-stabilizing microcapsules relative to the final topical composition.

The cosmetic or pharmaceutical active principles which may be used in the topical compositions of the present invention are all those whose activity or penetration is capable of being increased by a local supply of heat.

As examples of cosmetic or pharmaceutical principles which may be used according to the present invention, mention may be made of

anti-wrinkle agents such as retinol and its derivatives (acetate, palmitate or propionate), retinoids, n-octanoylsalicylic acid and hydroxy acids,

antibacterial and/or antifungal agents such as chlorhexidine, hexetidine, henamidine and benzalkonium chloride,

antiacne agents such as triclosan, azelaic acid, benzoyl peroxide and salicylic acid,

free-radical scavengers and/or detoxifying agents such as ascorbic acid and derivatives thereof, for instance magnesium ascorbyl phosphate, proteins and enzymes, for example superoxide dismutase (SOD), peroxidases such as lactoperoxidase and lactoferrin, catalase, proteases such as subtillisin and papain, lipases, uricase, peptides and their derivatives, ubiquinone and cytochrome C,

keratolytic agents such as α-hydroxy acids, β-hydroxy acids and α-keto acids, for instance salicylic acid and its derivatives,

tanning accelerators such as tyrosine derivatives, depigmenting active agents such as kojic acid, arbutin and derivatives thereof,

natural colorants extracted from plants, for instance chlorophylline, or extracted from animals, for instance cochineal carmine, or caramel,

self-tanning active agents such as dihydroxy-acetone and indoles,

liporegulators such as caffeine and theophylline,

moisturizers such as sorbitol, xylitol, urea and plant DNA,

antidandruff agents such as piroctone olamine and pyridine thione derivatives,

agents for preventing hair loss, such as minoxidil.

The topical compositions of the present invention may also contain suitable adjuvants such as solvents, preserving agents, pH regulators, anti-oxidants, sequestering agents, preserving agents, pigments and colorants, fillers, emollients, antifoams, fatty substances such as oils, waxes and pasty fatty substances, dispersants, silicones such as volatile or non-volatile oils, gums, waxes or pastes, fragrances, surfactants, plasticizers, soluble or dispersible film-forming polymers and thickening or gelling polymers.

The topical compositions of the present invention are, for example, in the form of a mask for the face or the hair, a massage product, a slimming composition, a moisturizing cream, an anti-wrinkle cream, an anti sun cream, a cleansing composition, a lotion, a poultice, a relaxing product or a hair-removing cream.

A subject of the present invention is also insoluble solid substrates impregnated with a topical composition as described above.

Specifically, the cosmetic or pharmaceutical compositions of the present invention may also be used to impregnate an insoluble solid substrate. The insoluble substrate may be chosen from the group comprising woven or nonwoven textile materials, foams, sponges, wadding, felts, beads or films. It may especially be a nonwoven textile substrate made of fibres of natural origin such as flax, cotton or silk fibres, or of synthetic origin such as cellulose, viscose or vinyl polymer fibres or fibres of polyesters, for instance poly(ethylene terephthalate), polyolefins, for instance polyethylene or polypropylene, polyamides, for instance Nylon® or acrylic polymers. Nonwoven materials are described, for example, in “Nonwoven Binding Methods & Materials” by Riedel, Nonwoven World, 1987. These substrates are obtained according to processes known in the technical field of preparing nonwovens.

This substrate may comprise one or more layers having identical or different properties, and which may provide, for example, elasticity or softness properties depending on the intended use. The substrates may comprise, for example, two parts having different elasticity properties, for instance those described in international patent application WO 99/13861, or may comprise a single layer with different densities, as described in document WO 99/25318, or alternatively may comprise two layers of different texture, for instance the substrates described, for example, in international patent application WO 98/18441.

The substrate may have any size or shape which is suitable for the intended application.

It generally has a surface area of between 0.005 m² and 0.1 m² and preferably between 0.01 m² and 0.05 m². It is preferably in the form of rectangular wipes or round compresses.

The final article comprising the substrate and the impregnation composition is generally in moist form, with a degree of impregnation, that is to say an amount of composition relative to the weight of the solid substrate, of between 200 and 1 000% and preferably between 250 and 350%.

The techniques for impregnating the substrates are well known in the art and are all applicable to the present invention. Usually, the impregnation composition is added to the substrate by one or more techniques such as immersion, coating or vaporization.

It is also possible to make an article (or wipe) in dry form either by removing the water from the composition after impregnating it onto the substrate, or by depositing onto the substrate a composition in dry form in the form of a powder, granule or film, by any known production method, such as welding and bonding together multilayers thermally or by means of ultrasound. In this last embodiment, the composition is dried by any known means, for example by atomization, lyophilization or another similar technique.

Moist wipes or dry wipes may thus be obtained, according to the intended use. The moist wipes may be used as is, whereas the dry wipes are moistened before use.

A subject of the invention is also cosmetic treatment processes using the heat-stabilizing microcapsules described above. These processes have in common the heating of the microcapsules up to a temperature above the melting point of the encapsulated crystalline compounds, the application of the heated microcapsules containing the molten encapsulated compound and the slow cooling of the composition(s) containing the microcapsules applied while hot. The cosmetic active principle may be in the composition containing the heat-stabilizing microcapsules, and it is then heated and applied at the same time as these microcapsules, but it is equally possible to envisage a separate, prior application to the active principle, followed by applying a second composition containing the heated heat-stabilizing microcapsules. This second embodiment is particularly advantageous for heat-sensitive active principles that do not tolerate prolonged exposure to a high temperature.

Consequently, in a first embodiment, the cosmetic treatment process comprises the following steps consisting

in heating a cosmetic composition containing at least one cosmetic active principle and microcapsules containing at least one crystalline compound with a heat of fusion (ΔH_{f}), measured by differential thermal analysis, of between 75 and 330 kJ/kg and a melting point of greater than or equal to 30° C., up to a temperature above the melting point of the said crystalline compound,

in applying the cosmetic composition to the area of skin to be treated,

in leaving the cosmetic composition to cool on contact with the skin for a time which is sufficient to obtain the desired cosmetic effect, and optionally

in removing the cosmetic composition.

Another embodiment of the cosmetic treatment process of the present invention comprises the steps consisting

in applying a cosmetic composition containing at least one cosmetic active principle to the area of skin to be treated,

in heating a second composition containing, in a cosmetically acceptable support, microcapsules containing at least one crystalline compound with a heat of fusion (ΔH^{f}), measured by differential thermal analysis, of between 75 and 330 kJ/kg and a melting point of greater than or equal to 30° C., up to a temperature above the melting point of the said crystalline compound,

in applying this second composition to the area of skin to be treated, over the first cosmetic composition,

in leaving the two compositions in contact with the skin for a time which is sufficient to obtain the desired cosmetic effect, and optionally

in cleaning the area of skin to be treated.

A third embodiment of the cosmetic treatment process of the present invention comprises the steps consisting

in applying to the area of the body or the face to be treated a cosmetic composition containing at least one cosmetic active principle and microcapsules containing at least one crystalline compound with a heat of fusion (ΔH^{f}), measured by differential thermal analysis, of between 75 and 330 kJ/kg and a melting point of greater than or equal to 30° C.,

in massaging the coated area so as to increase the local temperature up to a value which is sufficient to melt the crystalline compound,

in leaving the cosmetic composition to act and cool on contact with the skin for a time which is sufficient to obtain the desired cosmetic effect, and optionally

in removing the cosmetic composition.

These cosmetic treatment processes exploit the heat-storing capacity of the heat-stabilizing microcapsules described above. Specifically, during the cooling of the composition(s) applied to the area to be treated, the molten encapsulated compound recrystallizes and returns the latent heat of fusion absorbed during the prior heating of the microcapsules. This prolongation of the thermal effect increases the penetration of the active principle into the skin and/or its cosmetic activity.

### EXAMPLE 1

Massage gel

| | | | |
|---|---|---|---|
| A | Carboxyvinyl polymer (Sepigel ® 305, SEPPIC) | 0.2 | g |
| | Carboxyvinyl polymer (Pemulen ®, GOODRICH) | 1.5 | g |
| | Glycerol | 3 | g |
| | Demineralized water | 35 | g |
| | Triethanolamine | 0.5 | g |
| B | Silicone oil | 5 | g |
| | Squalane | 2 | g |
| | Silicone gum | 1 | g |
| | Fragrance | 0.2 | g |
| | Colorant | 0.02 | g |
| C | Ethyl alcohol | 20 | g |
| | Caffeine | 3 | g |
| | Demineralized water | 18.58 | g |
| D | Eicosane microcapsules (9850 Q, 3 M) | 10 | g |

### EXAMPLE 2

Cleansing composition

| Phase A | |
|---|---|
| Glyceryl stearate/PEG 100 stearate | 0.55% |
| (Arlacel ® 165) | |
| Cetyl alcohol | 0.15% |
| Xanthan gum | 0.1% |
| Isopropyl palmitate | 3.8% |
| Phase B | |
| Water | qs 100% |
| Preserving agent | qs |
| Phase C | |
| Heat-stabilizing microcapsules | 10% |

Phases A and B are heated separately up to a temperature of between 75 and 80° C. and phase A is then added to phase B with stirring. The stirring is continued for 5 minutes. Phase C is added with gentle stirring. The mixture is allowed to cool to room temperature with gentle stirring and wipes are impregnated with the emulsion obtained.

### EXAMPLE 3

Slimming wipes

| A lotion prepared from the ingredients | |
|---|---|
| below is used to impregnate wipes: | |
| Caffeine | 3% |
| Triethanolamine | 0.4% |
| Salicylic acid | 0.2% |
| Cetyl alcohol | 10% |
| Extract of butcher's-broom | 1% |
| Preserving agents | qs |
| Heat-stabilizing microcapsules | 10% |
| Water | qs 100% |

1. Topical composition comprising, in a physiologically acceptable support, a combination
of at least one cosmetic or pharmaceutical active principle and
of microcapsules containing at least one crystalline compound with a heat of fusion (ΔH_{f}), measured by differential thermal analysis, of between 75 and 330 kJ/kg and a melting point of greater than or equal to 30° C.

2. Cosmetic composition according to claim 1, characterized in that the microcapsules have a leaktight envelope which is impervious to the molten crystalline compound.

3. Composition according to claim 1 or 2, characterized in that the melting point of the encapsulated crystalline compound is between 35 and 45° C.

4. Composition according to one of the preceding claims, characterized in that the encapsulated crystalline compound has a heat of fusion of between 100 and 300 kJ/kg and preferably between 150 and 280 kJ/kg.

5. Composition according to one of the preceding claims, characterized in that the encapsulated crystalline compound is chosen from
aliphatic hydrocarbons containing from 13 to 28 carbon atoms,
aromatic hydrocarbons,
saturated or unsaturated C₉₋₂₄ fatty acids,
saturated, linear or branched C₁₄₋₃₆ fatty alcohols,
C₁₀₋₂₂ fatty acid esters,
mineral salts containing a large fraction of water of crystallization,
C₁₂₋₁₈ fatty acid triglycerides,
silicone waxes,
beeswax derivatives, and
crystalline hot-melt polymers.

6. Composition according to any one of the preceding claims, characterized in that the encapsulated crystalline compound is an aliphatic hydrocarbon with a linear chain containing from 19 to 28 carbon atoms and preferably from 20 to 23 carbon atoms.

7. Composition according to any one of the preceding claims, characterized in that the material forming the leaktight envelope is chosen from polyamides, polyurethanes, polyureas, polyesters, polycyanoacrylates, urea-formaldehyde or melamine-formaldehyde resins, gelatin/gum arabic systems and silica.

8. Composition according to any one of the preceding claims, characterized in that the microcapsules have a mean diameter of between 0.01 and 100 micrometres and preferably between 0.05 and 50 micrometres.

9. Composition according to any one of the preceding claims, characterized in that the microcapsules represent from 0.1% to 95% by weight and preferably from 5% to 90% by weight of the final topical composition.

10. Composition according to any one of the preceding claims, characterized in that the cosmetic or pharmacological active principle is chosen from
anti-wrinkle agents,
antibacterial and/or antifungal agents,
antiacne agents,
free-radical scavengers and/or detoxifying agents,
keratolytic agents,
tanning accelerators,
natural colorants extracted from plants or animals,
self-tanning active agents,
liporegulators,
moisturizers,
antidandruff agents,
agents for preventing hair loss.

11. Composition according to any one of the preceding claims, characterized in that it is a mask for the face or the hair, a massage product, a slimming composition, a moisturizing cream, an anti-wrinkle cream, an antisun cream, a cleansing composition, a lotion, a poultice, a relaxing product or a hair-removing cream.

12. Topical composition comprising, in a physiologically acceptable support, a combination
of at least one pharmaceutical active principle, and
of microcapsules with a leaktight envelope containing at least one crystalline compound with a heat of fusion (ΔH_{f}), measured by differential thermal analysis, of between 75 and 330 kJ/kg and a melting point of greater than or equal to 30° C., as a medicinal product.

13. Cosmetic treatment process comprising the steps consisting
in heating a composition according to any one of claims 1 to 11 containing at least one cosmetic active principle and microcapsules containing at least one crystalline compound with a heat of fusion (ΔH_{f}), measured by differential thermal analysis, of between 75 and 330 kJ/kg and a melting point of greater than or equal to 30° C., up to a temperature above the melting point of the said microcapsules,
in applying the cosmetic composition to the area of skin to be treated,
in leaving the cosmetic composition to cool on contact with the skin for a time which is sufficient to obtain the desired cosmetic effect, and optionally
in removing the cosmetic composition.

14. Cosmetic treatment process comprising the steps consisting
in applying a cosmetic composition containing at least one cosmetic active principle to the area of skin to be treated,
in heating a second composition containing, in a cosmetically acceptable support, microcapsules containing at least one crystalline compound with a heat of fusion (ΔH_{f}), measured by differential thermal analysis, of between 75 and 330 kJ/kg and a melting point of greater than or equal to 30° C., up to a temperature above the melting point of the said microcapsules,
in applying this second composition to the area of skin to be treated, over the first cosmetic composition,
in leaving the two compositions in contact with the skin for a time which is sufficient to obtain the desired cosmetic effect, and optionally
in cleaning the area of skin to be treated.

15. Cosmetic treatment process comprising the steps consisting
in applying to the area of the body or the face to be treated a cosmetic composition according to any one of claims 1 to 11 containing at least one cosmetic active principle and microcapsules containing at least one crystalline compound with a heat of fusion (ΔH_{f}), measured by differential thermal analysis, of between 75 and 330 kJ/kg and a melting point of greater than or equal to 30° C.,
in massaging the coated area so as to increase the local temperature up to a value which is sufficient to melt the crystalline compound,
in leaving the cosmetic composition to act and cool on contact with the skin for a time which is sufficient to obtain the desired cosmetic effect, and optionally
in removing the cosmetic composition.

16. Use of microcapsules containing at least one crystalline compound with a heat of fusion (ΔH_{f}), measured by differential thermal analysis, of between 75 and 330 kJ/kg and a melting point of greater than or equal to 30° C., for the manufacture of a product intended to improve the cutaneous activity and/or penetration of a pharmaceutical active principle applied simultaneously or beforehand.

17. Insoluble solid substrate impregnated with a composition according to any one of claims 1 to 12.

18. Insoluble solid substrate according to claim 17, characterized in that it is a nonwoven textile substrate made of fibres of natural or synthetic origin.

19. Insoluble solid substrate according to either of claims 17 and 18, characterized in that it has a surface area of between 0.005 m² and 0.1 m² and preferably between 0.01 m² and 0.05 m².

20. Insoluble solid substrate according to one of claims 17 to 19, characterized in that the degree of impregnation, that is to say the amount of cosmetic or pharmaceutical composition relative to the weight of the solid substrate, is between 200% and 1 000% and preferably between 250% and 350%.

21. Insoluble solid substrate according to one of claims 17 to 20, characterized in that it is in the form of rectangular wipes or round compresses.
